(19) United States
(12) Patent Application Publication
(10) Pub. No.: US 2010/0114153 A1
(43) Pub. Date: May 6, 2010

(54) Snap-Off Diet Aid™ tragus clip with single-point removable pressure head

(76) Inventors: John Michael Kretz, Lodi, OH (US); Paul Otto Kretz, North Royalton, OH (US)

Correspondence Address: Paul Kretz, P.O. Box 33851, North Royalton, OH, 44133, US

(21) Appl. No.: 12/584,784

(22) Filed: Sep. 11, 2009

Related U.S. Application Data
(60) Provisional application No. 61/096,788, filed on Sep. 13, 2008.

(51) Int. Cl. A61B 17/00

(52) U.S. Cl. 606/204

(57) ABSTRACT

This acupressure clip is designed to apply single-point pressure to the tragus of the ear to achieve either appetite control or a sense of tranquility. The clip has two opposing wing-shaped finger tabs joined by a reverse-compression band with an integral stop. The band applies pressure to the tragus through the single raised nub of the attached desired pressure head. The integral stop acts to prevent the band from passing the yield point of the material. The finger tabs have ridges to prevent slippage. Each of the interchangeable heads has a specifically molded nub located to activate a single acupressure point on the tragus of the ear for either appetite control or to achieve a sense of tranquility. At the end of the lower finger tab is a compression pad with an embossment to ensure proper fit and backing for the single-point pressure to be applied from the opposing head. The upper splayed pad is designed with a locking raised protrusion to secure and ensure proper location of the desired head. The heads lock into the upper splayed pad with a corresponding locator hole to secure the desired head during use.

### CROSS REFERENCE TO RELATED APPLICATIONS

This application is a continuation of U.S. Provisional patent application Ser. No. 61/096,788 which was filed on Sep. 13, 2008 and the description of which is incorporated herein by reference.

### REFERENCES CITED

U.S. Patent Documents

| US Patent | Issued to | Date |
|---|---|---|
| 3,923,064 | Leupold | December 1975 |
| 4,073,296 | McCall | February 1978 |
| 4,319,574 | Sun | March 1982 |
| 4,479,495 | Isaacson | October 1984 |
| 5,366,475 | Voss et al | November 1994 |
| 5,662,679 | Voss et al | September 1997 |

### RELATED U.S. APPLICATION DATA

Provisional Application Number 61/096,788 Filed on Sep. 13, 2008

### BACKGROUND OF INVENTION

The Snap-Off Diet Aid™ contemporary methods of plastics design and manufacturing with an ancient method of Chinese healing called “acupressure.” Acupressure is a highly developed treatment modality, which has been used for centuries by Chinese healers. Over time, those healers discovered that, when pressure was accurately applied by their fingers or specially made devices, to specific areas on the human body, often for varying periods of time the physical discomfort and suffering brought about by many acute and chronic human illnesses and diseases were often alleviated. These specific areas of the body, among them the hands, wrists, and ears, are known as “pressure points.” Some of those “pressure points” were found to provide relief from the symptoms of chronic conditions such as diabetes and arthritis.

A pressure point located on the tragus of the ear was discovered to be associated with appetite suppression. Pressure applied at this point can be utilized to assist people who are trying to lose weight. The Snap-Loss Diet Aid™ was developed to exert continuous pressure on this relatively small area of the body (approximately 0.080″ x 0.080″). While the Snap-Off Diet Aid™ is not intended as a singular method for weight reduction, it can be an integral tool in the successful dieter's weight loss program.

### SUMMARY OF INVENTION

The main advantage of the Snap-Off Diet Aid™ over other similar products is that the user is able to position the Snap-Off Diet Aid™ more accurately, as directed, on the tragus of the ear. The result is a more consistent, focused and efficacious compression of a single pressure point on one side of the tragus as opposed to equal pressure on both sides.

A second advantage of the Snap-Off Diet Aid™ over similar products is that it is constructed of homogenous injection molded plastic, such as polycarbonate (PC). This material remains flexible from −148° F. and solid to +275° F. without losing its elasticity or breaking. Furthermore, due to its design and construction, the Snap-Off Diet Aid™ will retain its pressure over a longer period of time. The heads are made of polyvinyl chloride (PVC) material which remains flexible from −34.6° F. to +250° F.

Another advantage of the Snap-Off Diet Aid™ over similar products is that the clip with the point on one side simulates the effect of a needle in acupuncture in a more comfortable and practical fashion. The clip does not puncture, but achieves an indistinguishable effect.

A final advantage of the Snap-Off Diet Aid™ over similar products is that the pads with the specific pressure heads are easily removable and interchangeable without the need for an extra tool. This also allows for more point-specific heads to be added at a later time.

### DESCRIPTION OF THE DRAWINGS

FIG. 1 Assembly sketch of Tranquil pressure head for the Snap-Off Diet Aid.

FIG. 2 Assembly sketch of Weight pressure head for the Snap-Off Diet Aid.

FIG. 3 Dimensional drawing of clip without pressure heads.

FIG. 4 Clearance distance for clip in resting state.

FIG. 5 Dimensional drawing of pressure heads.

FIG. 6 Demonstrative illustration of the clip on an ear.

FIG. 7 Demonstrative illustration of compression of clip by finger and thumb

FIG. 8 Illustration of the human ear.

FIG. 9 Exploded view of the clip and the pressure heads.

### DETAILED DESCRIPTION

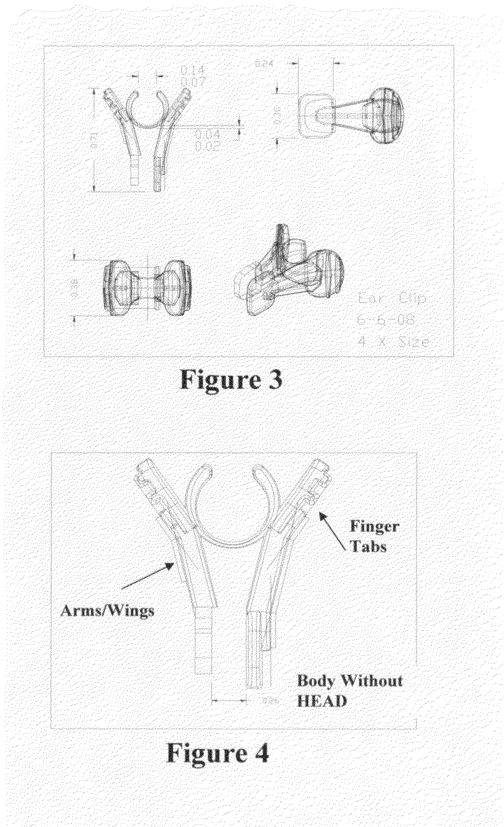

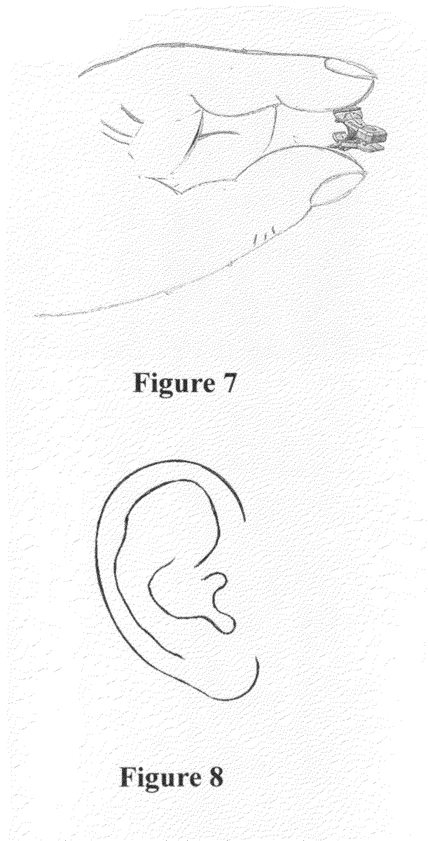

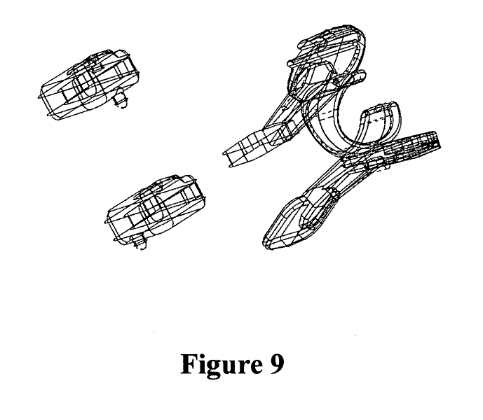

The Snap-Off Diet Aid™ consists of upper and lower arms, as depicted in FIG. 3, each end flared with corners rounded and the surfaces crossed width-wise by two raised, embossed ridges, which provide anchors for a finger and thumb, as depicted in FIG. 7.

Two connecting arms, one upper and one lower, detailed in FIGS. 3 and 4, each connected to the tapered end of the corresponding tabs, described in the immediately preceding paragraph.

Both upper and lower areas are reinforced by increasing the thickness of the arm with a raised plane running the length of the load-bearing surfaces on the arms (FIG. 3).

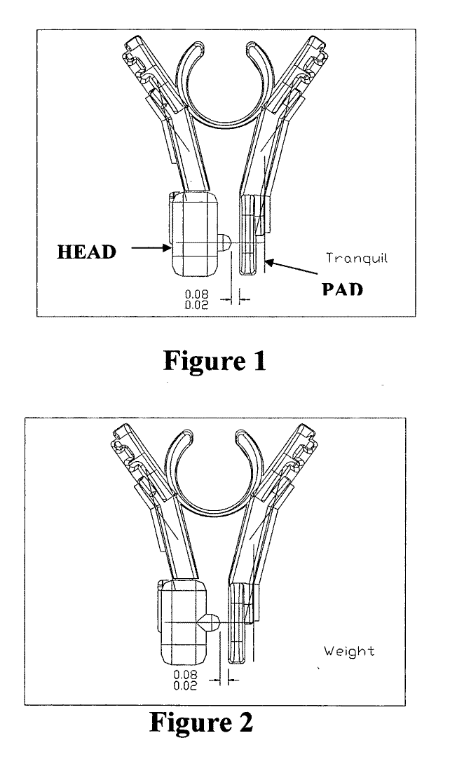

The effect point is located by centering the head of the clip on the tragus thus locating the correct point for the nub to apply pressure for the desired affect. By varying the location of the nub on the different heads, different pressure points are stimulated, thus achieving different effects (FIGS. 1, 2 & 6).

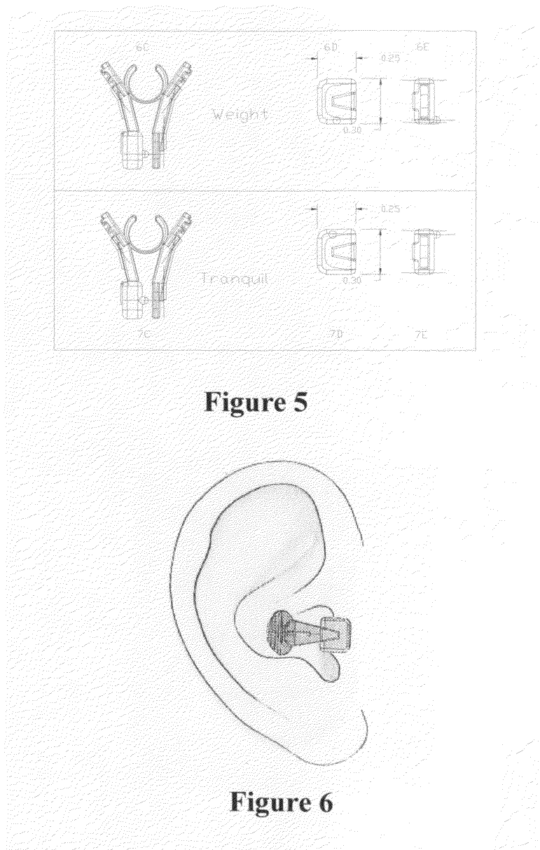

An interchangeable upper acupressure pressure head (FIG. 5), consisting of a single raised embossed dimple 0.0625″ higher then the head itself applies pressure on the single acupressure point required for the desired effect. A lower retainer compression pad provides back support for the compression exerted by the upper acupressure splayed pad with fitted compression head. In addition, it maintains proper position of the Snap-Off Diet Aid™ on the tragus of the ear. This pad also ensures proper pressure to be applied by the nub of the selected pressure head from the opposing side.

When appropriate head is attached, the pressure on the side with the interchangeable head is focused on the appropriate pressure point to give the desired response. The interchangeability of acupressure pads allows for the future addition of other pads to stimulate other known points.

The heads being claimed suppress appetite and reduce stress.

We claim:

1. A tragus acupressure clip comprising:
a. A single injection-molded apparatus with two opposing finger tabs connected by a compression band that stands 71 degrees (plus or minus 2 degrees) in its resting state,
b. Two compression pads on opposite ends of the finger tabs.

2. A tragus acupressure clip described in claim one made of polycarbonate or equivalent material.

3. A tragus acupressure clip described in claim one comprising of compression pads that allow for pressure heads to slip onto compression pads without fasteners.

4. A tragus acupressure clip with removable pressure heads described in claim three comprising of:
a. One compression pad with locking raised protrusion to ensure and secure proper location of pressure heads,
b. Removable pressure heads with corresponding locator holes to secure pressure heads during use.

5. A tragus acupressure clip with removable pressure heads described in claim three comprising of compression pads with appropriate thickness to ensure 0.020″ to 0.080″ minimum separation in closed state for effective pressure at the appropriate points.

6. A tragus acupressure clip with removable pressure heads described in claim three comprising of an incorporated physical positive stop of compression band to prevent material from exceeding physical yield point to ensure retained tension of the compression band for necessary effect of the pressure heads.

7. A tragus acupressure clip with removable pressure heads described in claim three comprising a pressure head with raised nub in specific location that stimulates the pressure point on the ear to suppress appetite (FIG. 2).

8. A tragus acupressure clip with removable pressure heads described in claim three comprising a pressure head with raised nub in specific location that stimulates the pressure point on ear to reduce stress (FIG. 1).

9. A tragus acupressure clip with finger tabs consisting of:
a. Finger tabs reduced at acute angles to aid user in maintaining control of clip at all points of compression when applying to tragus of ear, and
b. Raised grip lines on finger tabs to reduce slippage in fingers of user when compressing the clip.
